(19) United States
(12) Patent Application Publication
(10) Pub. No.: US 2007/0185230 A1
(43) Pub. Date: Aug. 9, 2007

(54) Reactive oligomeric thiol and ene materials as dental restorative mixtures

(75) Inventors: Christopher N. Bowman, Boulder, CO (US); Carioscia Jacquelyn, Broomfield, CO (US); Hui Lu, Boulder, CO (US); Jeffrey W. Stansbury, Aurora, CO (US)

Correspondence Address: GREENBERG TRAURIG, LLP, 1200 SEVENTEENTH STREET, SUITE 2400, DENVER, CO, 80202, US

(73) Assignee: The Regents of the University of Colorado CU Technology Transfer Office, Boulder, CO (US)

(21) Appl. No.: 10/598,641

(22) PCT Filed: Mar. 8, 2005
(86) PCT No.: PCT/US05/07938

§ 371 Date: Sep. 7, 2006

Related U.S. Application Data
(60) Provisional application No. 60/551,688, filed on Mar. 9, 2004.

(51) Int. Cl. A61K 6/083

(52) U.S. Cl. 523/115

(57) ABSTRACT

The present invention provides a dental composition comprising a curable blend of one or more polythiol compounds and one or more polyvinyl compounds; where one or both compounds are oligomers. In one aspect, the polythiol compounds are polythiol oligomers formed by prepolymerization of polyvinyl monomers in the presence of an excess of polythiol monomers. In another aspect, the polyvinyl compounds are polyvinyl oligomers formed by prepolymerization of polythiol monomers in the presence of an excess of polyvinyl monomers. The dental composition may further comprise one or more fillers or photoinitiators known in the art. The invention also comprises methods of making a dental prosthesis comprising the composition described above. Use of the thiol-ene oligomeric system results in cured (polymerized) dental compositions having improved physical properties, including low-shrinkage properties and reduced shrinkage induced-stress, enhanced double bond conversion percentage, and reduced odor.

This application is being filed as a PCT International Patent application on 8 Mar. 2005, in the name of Regents of the University of Colorado, a U.S. national university, applicant for the designation of all countries except the US, and Christopher N. Bowman, Jacquelyn Carioscia, and Jeffrey W. Stansbury, all U.S. citizens, and Hui Lu, citizen of the PR China; applicants for the designation of the US only, and claims priority to U.S. Application Ser. No. 60/551,688 filed 9 Mar. 2004.

Statement Regarding Federally Sponsored Research or Development

The invention was sponsored by NIH Grant No. DE 10959 and the government has certain rights to this invention.

### FIELD OF THE INVENTION

The present invention relates to a thiol-ene polymer system with low shrinkage and more particularly to a curable thiol-ene polymer system exploiting prepolymerization for use as a dental restorative resin.

### BACKGROUND DESCRIPTION OF THE RELATED ART

Currently, commercial photoactivated dental restorative resins are based on dimethacrylates where the reaction mechanism is achieved through chain-growth free radical polymerization. Existing dimethacrylate systems are popular for fillings and other dental prostheses because of their esthetic merit and “cure-on-command” feature.

The photoactivated restorative materials are often sold in separate syringes or single-dose capsules of different shades. If provided in a syringe, the user dispenses (by pressing a plunger or turning a screw adapted plunger on the syringe) the necessary amount of restorative material from the syringe onto a suitable mixing surface. Then the material is placed directly into the cavity, mold, or location of use. If provided as a single-dose capsule, the capsule is placed into a dispensing device that can dispense the material directly into the cavity, mold, etc. After the restorative material is placed, it is photopolymerized or cured by exposing the restorative material to the appropriate light source. The resulting cured polymer may then be finished or polished as necessary with appropriate tools. Such dental restoratives can be used for direct anterior and posterior restorations, core build-ups, splinting and indirect restorations including inlays, onlays and veneers.

Although easy to use, these systems have several drawbacks, primarily associated with the polymerization volume shrinkage and shrinkage stress, and poor conversion of the dimethacrylate systems' monomers into polymer. The current systems can only reach a final double bond conversion of 55 to 75%, which not only contributes to the insufficient wear resistance and mechanical properties, but also jeopardizes the biocompatibility of the composites due to the leachable, unreacted monomers. Dimethacrylate based resins exhibit significant volumetric shrinkage during polymerization. This induced shrinkage causes stress, which results in tooth-composite adhesive failure, microleakage and recurrent dental caries, significantly reducing the longevity and utility of current dental restorative composite. Furthermore, as one tries to increase the final double bond conversion to reduce the unreacted monomers, the volumetric shrinkage and shrinkage stress unfortunately also increase, which has been a persistant problem since the development of this class of resins.

Thus, the need exists for dental compositions that exhibit low shrinkage, low shrinkage stress, and high conversion during curing to improve the longevity and utility of dental restorative composites.

### SUMMARY OF THE INVENTION

The present invention provides a dental composition comprising a curable blend of one or more polythiol compounds and one or more polyvinyl compounds; where one or both compounds are oligomers. In one aspect, the polythiol compounds are polythiol oligomers formed by prepolymerization of polyvinyl monomers in the presence of an excess of polythiol monomers. In another aspect, the polyvinyl compounds may be polyvinyl oligomers formed by prepolymerization of polythiol monomers in the presence of an excess of polyvinyl monomers. The dental composition may further comprise one or more fillers or photoinitiators known in the art. The invention also comprises methods of making a dental prosthesis comprising the composition described above. Use of the thiol-ene oligomeric system results in cured (polymerized) dental compositions having improved physical properties, including low-shrinkage properties and reduced shrinkage induced-stress, enhanced double bond conversion percentage, and reduced odor.

### BRIEF DESCRIPTION OF THE DRAWINGS

FIG. 1 shows a flow chart illustrating a method of obtaining a dental prosthesis utilizing an oligomeric thiol-ene polymer system.

FIG. 2 shows functional group conversion as a function of time for preparation of thiol-terminated oligomers using simultaneous FTIR monitoring of both the thiol and ene peaks: tetrathiol terminated oligomer using tetrathiol (●):Triazine Triallyl (◯) reacted in a ˜6.6:1 monomer functionality ratio, and trithiol terminated oligomer using trithiol (▪):Triazine Triallyl (×) reacted in a ˜4.4:1 monomer functionality ratio. The UV light intensity was 80 mW/cm², and 0.1 wt % DMPA was used as the initiator.

FIG. 3 shows conversion of the vinyl functional group for Trithiol/Trivinyl (M:M), Trithiol/Trivinyl Oligomer (M:O), Trithiol oligomer/Trivinyl oligomer (O:O), and Bis-GMA/TEGDMA (70/30 by wt.) as a function of irradiation time;0.1 wt % DMPA; UV=15 mW/cm2. The thiol-ene monomer mixture was prepared to have an equivalent concentration of the two functional groups.

FIG. 4 illustrates T_{g} loss tangent peaks for thiol-ene systems trithiol/triazine triallyl and tetrathiol/triazine triallyl compared to Bis-GMA/TEGDMA.

FIG. 5 shows shrinkage stress as a function of conversion for Bis-GMA/TEGDMA (70/30 wt %) (−−) and (−) Tetrathiol/Triazine Triallyl and (−) Tetrathiol oligomer/Triazine Triallyl, cured using 400 W/cm² visible light and 0.3 wt % CQ and 0.8 wt % EDAB as coinitiators, for 1 minute at room temperature.

FIG. 6 shows shrinkage stress as a function of double bond conversion of Trithiol and Trithiol oligomer reacted with Triazine Triallyl, cured with UV=17 mW/cm² for 50 seconds at room temperature.

FIG. 7 shows percent volume shrinkage for Trithiol/Triallyl, Trithiol/Trivinyl, Trithiol/Trivinyl oligomer, and Trithiol oligomer/Trivinyl oligomer systems as a function of time; 0.1 wt % DMPA, UV=15 mW/cm². All mixtures were prepared to have an equivalent concentration of the two functional groups.

FIG. 8A shows actual thiol and ene conversion for several thiol-ene systems.

FIG. 8B shows actual percent volume shrinkage for several thiol-ene systems.

FIG. 9 shows percent volume shrinkage for Trithiol/Triazine Triallyl, Trithiol oligomer/Triazine Triallyl, Tetrathiol/Triazine Triallyl, and Tetrathiol oligomer/Triazine Triallyl systems as a function of time; 0.1 wt % DMPA, UV=15 mW/cm². All mixtures were prepared to have an equivalent concentration of the two functional groups.

### DETAILED DESCRIPTION OF THE INVENTION

The present invention relates to a dental restorative composition with improved properties comprising a curable oligomeric thiol-ene polymer system. Use of the thiol-ene oligomeric system results in cured (polymerized) dental compositions having improved physical properties, including low-shrinkage properties, reduced shrinkage induced-stress, and enhanced double bond conversion percentage when compared to currently available commercial photoactivated dental restorative resins. In addition, oligomeric thiol-ene systems have reduced odor when compared to monomeric thiol-ene systems.

The oligomeric thiol-ene polymer system comprises a curable blend of one or more polythiol compounds and one or more polyvinyl compounds; where one or both compounds are oligomers. The oligomeric thiol-ene polymer system utilizes prepolymerization of polythiol monomers with polyvinyl monomers, with one monomer in excess, to obtain non-gelled polythiol or polyvinyl functionalized oligomers. The polythiol functionalized oligomers are further combined with either polyvinyl monomers or polyvinyl oligomers in amounts such that a stoichiometric equivalent number of thiol and vinyl functional groups are present. Alternatively, polyvinyl oligomers may be combined with polythiol monomers or polythiol oligomers in amounts such that a stoichiometric equivalent number of vinyl and thiol functional groups are present. This combination of oligomer-monomer or oligomer-oligomer is defined as the oligomeric thiol-ene polymer system.

Current dental resins react via a chain growth mechanism, where as the proposed oligomeric thiol-ene systems react via a step growth mechanism, which allows for the novel oligomerization (prepolymerization) of thiol and ene materials.

Building on the advantages of the step-growth mechanism, it is possible to oligomerize (prepolymerize) thiol and ene monomers, achieving a higher extent of polymerization prior to formulating the final resin and completing the polymerization in the restoration. This will decrease the functional group concentration, more specifically the vinyl functional group concentration, which is responsible for shrinkage, thus creating an even lower shrinkage material than the dimethacrylate and monomeric thiol-ene systems, while maintaining mechanical integrity. Higher functional group conversion also results in less extractable monomer. Furthermore, oligomerization of thiol and ene materials reduces or eliminates low molecular weight reactants responsible for odor, as well as the amount of extractable monomer in the resin, thus reducing the cytotoxicity of the resin. Glass transition temperatures (Tg), determined by dynamic mechanical analysis (DMA), for oligomeric thiol-ene systems have a narrower glass transition peak width indicating that oligomeric thiol-ene systems result in more homogenous networks than conventional Bis-GMA/TEGDMA systems.

Further beneficial characteristics of dental compositions comprising thiol-ene resins are a demonstrated lack of oxygen inhibition and the possibility of a photoinitiator free system (Cramer and Bowman, (2001). Journal of Polymer Science, Part A: Polymer Chemistry, 39:3311-3319).

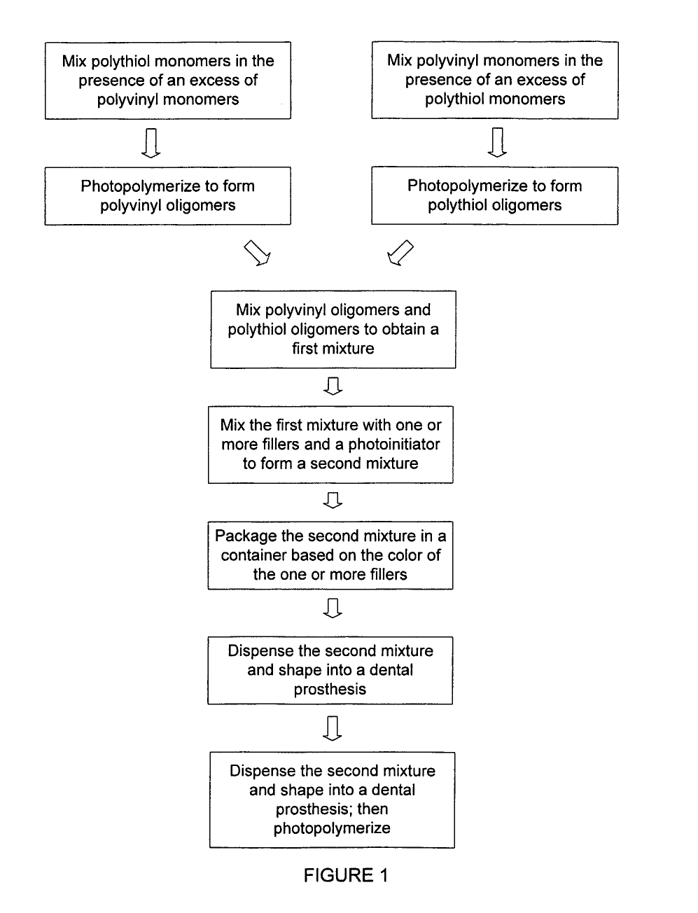

Embodiments of the present invention comprise an oligomeric thiol-ene polymer system which employs prepolymerization. A preferred embodiment utilizes a method of providing a dental composition comprising the oligomeric thiol-ene system, illustrated in FIG. 1. Embodiments of the curable thiol-ene system preferably have about 45%-55% of functional groups as thiol functional groups. The balance of the functional groups in the system may be vinyl functional groups. In preparation of the curable thiol-ene systems, because of the step growth mechanism of the polymerization, for highest conversion it is preferred to have approximately equal amounts of functional groups (i.e., 50% thiol (—SH) functional groups and 50% vinyl (CH═CH₂) functional groups).

In addition to thiols and vinyl functional groups, in some embodiments additional functional groups may be provided to tailor and provide additional properties.

Thiol bearing monomers suitable for embodiments of the present invention include any monomer with a discrete chemical formula having at least one thiol (mercaptan or “—SH”) functional group. Thiols are any of various organic compounds having —SH functional group which are analogous to alcohols but in which sulfur replaces the oxygen of the hydroxyl group. Examples of suitable thiol bearing monomers include: 1-Octanethiol; and Butyl 3-mercaptopropionate. Polythiol monomers suitable for embodiments the present invention further include any monomer having at least two thiol (mercaptan or “—SH”) functional groups. Suitable polythiol monomers have a discrete chemical formula and may have at least two functional thiol groups, more preferably at least three thiol functional groups, and be of any molecular weight. Examples of suitable commercially available polythiol bearing monomers include: pentaerythritol tetrakis(3-mercaptopropionate) (tetrathiol, PETMP); trimethylol tris(3-mercaptopropionate) (trithiol); 1,6-hexanedithiol.

Polyvinyl monomers having “-ene,” or vinyl, functional groups suitable for embodiments of the present invention include any monomer having a discrete chemical formula and having one or more vinyl functional groups, i.e., reacting “—CH═CH₂” groups. Polyvinyl monomers suitable for the present invention have at least two, but more preferably at least three, vinyl functional groups. The vinyl groups may be provided by allyls, allyl ethers, vinyl ethers, acrylates or other monomers containing vinyl groups. Examples of suitable commercially available polyvinyl monomers include: Trimethylolpropane trivinyl ether (trivinyl); Pentaerythritoltriallyl ether (triallyl); 1,3,5-Triallyl-1,3,5-triazine-2,4,6-trione (triazine triallyl, TATATO).

Access to additional polythiol monomers and polyvinyl monomers may be obtained by the reaction of a diisocyanate in the presence of an excess of an alcohol monomer to form a polyalcohol compound. Diisocyanates of the formula O═C═N—R—N═C═O, where R may be aliphatic, alkenyl, alkynyl, alkoxyalkyl, aryl, aralkyl, aryloxyaryl, or aralkoxy.

The term “aliphatic” or “aliphatic group” as used herein means a straight-chain or branched C₁₋₁₂ hydrocarbon chain that is completely saturated or that contains one or more units of unsaturation, or a monocyclic C₃₋₈ hydrocarbon or bicyclic C₈₋₁₂ hydrocarbon that is completely saturated or that contains one or more units of unsaturation, but which is not aromatic (also referred to herein as “carbocycle” or “cycloalkyl”), that has a single point of attachment to the rest of the molecule where in any individual ring in said bicyclic ring system has 3-7 members. For example, suitable alkyl groups include, but are not limited to, linear or branched or alkyl, alkenyl, alkynyl groups and hybrids thereof such as (cycloalkyl)alkyl, (cycloalkenyl)alkyl or (cycloalkyl)alkenyl.

The terms “alkoxy,” “hydroxyalkyl,” “alkoxyalkyl” and “alkoxycarbonyl,” used alone or as part of a larger moiety include both straight and branched chains containing one to twelve carbon atoms. The terms “alkenyl” and “alkynyl” used alone or as part of a larger moiety shall include both straight and branched chains containing two to twelve carbon atoms.

The term “heteroatom” means nitrogen, oxygen, or sulfur and includes any oxidized form of nitrogen and sulfur, and the quaternized form of any basic nitrogen. The term “aryl” used alone or in combination with other terms, refers to monocyclic, bicyclic or tricyclic carbocyclic ring systems having a total of five to fourteen ring members, wherein at least one ring in the system is aromatic and wherein each ring in the system contains 3 to 8 ring members. The term “aryl” may be used interchangeably with the term “aryl ring”. The term “aralkyl” refers to an alkyl group substituted by an aryl. The term “aralkoxy” refers to an alkoxy group substituted by an aryl.

In preferred embodiments, diisocyanates of the formula O═C═N—R—N═C═O, where R may be —(CH₂)₄—, —(CH₂)₁₂—, —(CH₂)₆—, —(CH₂)₃CH(CH₃)CH₂—, —(CH₂)₈—, —C₆H₄—, or —C₆H₃(CH₃)— may be utilized.

Alcohol monomers are defined as any compound having a discrete chemical formula with at least one alcohol (hydroxy, R′—OH) functional group; more preferably at least three hydroxyl groups, where R′ may be defined as may be aliphatic, alkenyl, alkynyl, alkoxyalkyl, aryl, aralkyl, aryloxyaryl, or aralkoxy. The alcohol monomer may also include other heteroatoms. In another preferred embodiment, the alcohol monomer also has at least one thiol (—SH) functional group.

The resultant polyalcohol compounds may subsequently be converted eitherto vinyl ethers (or other vinyl functionalities) to form polyvinyl monomers or to thiols to form polythiol monomers by synthetic means documented elewhere (Okimoto, et al. J. Am. Chem. Soc., 124:1590-1591(2002); Krishnamurthy and Aimino, J. Org. Chem. 54(18):4458-4462(1989)).

Vinyl ether conversion of polyalcohol compounds may be performed with vinyl acetate in the presence of an iridium complex catalyst (Okimoto et al., 2002). This strategy allows access to the oligomerization process with a greater variety of chemical structures. The same oligomeric products can be derivatized with both vinyl and thiol functional groups (in two separate batches) to facilitate miscibility that might not otherwise be possible.

Polythiol oligomers and polyvinyl oligomers are defined as non-gelled prepolymers and may be formed by prepolymerization of one functional group monomer in the presence of an excess of the other functional group monomer. For example, polythiol oligomers are formed by prepolymerization of polyvinyl monomers in the presence of an excess of polythiol monomers, such that the resultant non-gelled oligomer has a plurality of thiol functional groups. Polyvinyl oligomers are formed by prepolymerization of polythiol monomers in the presence of an excess of polyvinyl monomers, such that the resultant polyvinyl oligomer has a plurality of vinyl functional groups. The relative amounts of polythiol monomer and polyvinyl monomer used in may be described by the step growth polymerization gelation equation;

$$α = \frac{1}{\sqrt{r ( fa - 1 ) \cdot ( fb - 1 )}} \qquad ( Equation 1 )$$

where alpha is the fractional conversion at the gel point, fₐ and f_{b} are the weight average functionalities of the two comonomers and r is defined as the stoichiometric imbalance, or Nₐ/N_{b} (where Nₐ and N_{b} are the molar equivalents of each monomer present with N_{b>N}ₐ) (Odian, Principles of Polymerization, John Wiley and Sons, New York (1991)). While a crosslinked polymer is formed when alpha is less than one, non-gelled oligomer results if alpha is greater than one, i.e. that specific stoichiometric ratio will not gel even when all the limiting functional group has reacted. Hence, prereacting thiol-enes with a sufficient excess of one monomer, produces soluble, highly functional reactive thiol or vinyl oligomers (U.S. Pat. No. 5,459,175).

A polythiol compound is defined as either a polythiol oligimer or a polythiol monomer, as described above.

A polyvinyl compound is defined as either a polyvinyl oligomer or a polyvinyl monomer, as described above.

A thiol-ene curable composition (thiol-ene system) is defined as a blend comprising at least one polythiol compound and at least one polyvinyl compound wherein at least one compound is an oligomer. The ratio of thiol to vinyl functional groups in the thiol-ene system may vary from 55:45 to 45:55 thiol/vinyl. It is preferred that the ratio of thiol to vinyl function groups to be 50:50 thiol/vinyl.

In preferred embodiments, for polythiol oligomerization processes, an excess of thiol monomer was used, such that alpha (Equation (1)) was equal to 1.05, creating nearly exclusively thiol terminated reactive oligomers. Similarly, for all vinyl oligomerization processes an excess of vinyl monomer was used, such that alpha was equal to 1.05, creating nearly exclusively vinyl terminated reactive oligomers. In a preferred embodiment, specifically, a ˜4.4:1 monomer functionality ratio of thiol to ene in the trithiol:triazine triallyl thiol terminated oligomer, and ˜4.4:1 monomer functionality ratio of ene to thiol in the trithiol:trivinyl and trithiol:triallyl vinyl oligomers was used. A ˜6.6:1 monomer functionality ratio of thiol to ene in the tetrathiol: triazine triallyl thiol oligomerization was used.

Thiol-ene systems may also include and/or utilize various initiators, fillers, and accelerators depending on the applicationInitiators are defined as polymerization initiators, or photoinitiators.

Suitable polymerization initiators are those conventional initiators known in the art. For example, visible light curable compositions employ light-sensitive compounds such as benzil diketones, and in particular, DL-Camphorquinone (CQ) in amounts ranging from about 0.05 to about 0.5 weight percent (wt %). In a preferred embodiment, 0.3 wt % CQ is used as an initiator for visible light experiments, along with 0.8 wt % ethyl 4-(dimethylamino)benzoate (commonly known as EDMAB or EDAB).

Alternatively, for ultraviolet (UV) photopolymerization, 2,2-Dimethoxy-2-phenylacetophenone (DMPA) may be used as an initiator. In a preferred embodiment, 0.1 wt % DMPA is used as the initiator for UV light curing experiments.

Amine accelerators may be used as polymerization accelerators, as well as other accelerators. Polymerization accelerators suitable for use are the various organic tertiary amines well known in the art. In visible light curable compositions, the tertiary amines are generally acrylate derivatives such as dimethylaminoethyl methacrylate and, particularly, diethylaminoethyl methacrylate (DEAEMA), EDAB and the like, in an amount of about 0.05 to about 0.5 wt %. The tertiary amines are generally aromatic tertiary amines, preferably tertiary aromatic amines such as EDAB, 2-[4-(dimethylamino)phenyl]ethanol, N, N-dimethyl-p-toluidine (commonly abbreviated DMPT), bis(hydroxyethyl)-p-toluidine, triethanolamine, and the like. Such accelerators are generally present at about 0.5 to about 4.0 wt % in the polymeric component. In a preferred embodiment, 0.8 wt % EDAB is used in visible light polymerization. Certain embodiments of the thiol-ene system can be readily initiated by camphorquinone alone, without the presence of the amine accelerator. This is largely beneficial to the biocompatibility of photo-cured dental composites since studies have shown that certain tertiary amine accelerators, such as N,N-dimethyl-p-toluidine, are carcinogenic and mutagenic.

The dental compositions comprised of restorative materials may be unfilled, filled, or partially filled. The filled compositions can include many of the inorganic fillers currently used in dental restorative materials, the amount of such filler being determined by the specific function of the filled materials. Thus, for example, the resinous compositions are present in amounts of about 10 to about 40 weight percent of the total composition, and the filler materials are present in amounts of about 60 to about 90 weight percent of the total composition. Typical compositions for crown and bridge materials are about 25 percent by weight of the resinous material and about 75 percent by weight of the filler.

Dental restorative materials may be mixed with 45 to 85% by weight (wt %) silanized filler compounds such as barium, strontium, zirconia silicate and/or amorphous silica to match the color and opacity to a particular use or tooth. The filler is typically in the form of particles with a size ranging from 0.01 to 5.0 micrometers.

Other suitable fillers are known in the art, and include those that are capable of being covalently bonded to the resin matrix itself or to a coupling agent that is covalently bonded to both. Examples of suitable filling materials include but are not limited to, silica, silicate glass, quartz, barium silicate, strontium silicate, barium borosilicate, strontium borosilicate, borosilicate, lithium silicate, lithium alumina silicate, amorphous silica, ammoniated or deammoniated calcium phosphate and alumina, zirconia, tin oxide, and titania. Particularly suitable fillers are those having a particle size in the range from about 0.1 to about 5.0 micrometers, mixed with a silicate colloid of about 0.001 to about 0.07 micrometers. Some of the aforementioned inorganic filling materials and methods of preparation thereof are disclosed in U.S. Pat. No. 4,544,359 and U.S. Pat. No. 4,547,531, pertinent portions of which are incorporated herein by reference. The above described filler materials may be combined with a variety of composite forming materials to produce high strength along with other beneficial physical and chemical properties. Preferably, the filler is mixed with a resinous material to form high-strength dental composites. Suitable resin materials include those mentioned herein. A preferred resin comprises a curable oligomeric thiol-ene system described herein.

Conversion is defined as the loss of thiol or vinyl functional groups upon polymerization, or prepolymerization. Specifically, upon polymerization, the double-bond of the vinyl group (-ene, —CH═CH₂) is converted to a saturated ethane (-ane, —CH₂—CH₂—). The conversion of thiol (—SH) groups to thiol ethers (—S—CH₂—) occurs upon polymerization. Polymerization kinetics of thiol-ene systems may be monitored by Infrared spectroscopy (IR). Fourier Transform IR (FTIR) (e.g. Magna 750, Nicolet Instrument Corp., Madison, Wis.) may used to study the polymerization kinetics of the thiol-ene materials because of its inherent advantage of being able to measure the thiol and vinyl conversions simultaneously and rapidly (Cramer et al., J. Polymer Sci., Part A Polymer Chem., 39: 3311-3319 (2001)). For example, the infrared peak absorbance at 1643 cm⁻¹ may be used for determining the allyl group conversion; the peaks at 1619 and 1636 cm⁻¹ for vinyl ethers; and the peak at 2572 cm⁻¹ may be used for the thiol group conversion. Conversions may be calculated with the ratio of peak areas to the peak area prior to polymerization.

In addition to conversion kinetics, multiple material property measurements may be conducted. Samples for dynamic mechanical analysis (DMA) may be tested on, for instance, a DMA7e, Perkin-Elmer, Norwalk, Conn. DMA studies may be conducted over a temperature range of, for example, −50 to 120° C., with a ramping rate of 5° C./min using extension mode (sinusoidal stress of 1 Hz frequency) and the loss tangent peak was monitored as a function of temperature. The loss tangent is defined as the polymer's loss modulus divided by storage modulus. During a DMA test, loss tangent peak corresponds to the viscoelastic relaxation of polymer chain or segments. Normally, the largest loss tangent peak can be associated with the polymer's glass transition peak and the temperature of the loss tangent peak maximum was used to define T_{g} (glass transition temperature).

Dental restorations may be exposed to temperatures within a 0-60° C. range in the oral environment. If the temperature range approaches that of the T_{g} of the resin, this could cause a decrease in th e mechanical properties of the resin, ultimately leading to premature failure. In addition, resin homogeneity plays a role in how the mechanical properties of the resin are affected by the temperature change. A wide T_{g} peak signifies a lack of homogeneity, or more specifically a distribution of chain mobility. The maxima of the tan delta peak (often taken as the Tg) is only an average value, and thus if the oral environment reaches a temperature at which some of the chains below the average T_{g} become mobile, the mechanical properties of the system may be negatively affected.

Gel point conversion is defined as the point at which the resin becomes an infinite gel network.

The thiol-ene systems of the present invention have significant and unique advantages compared with (meth)acrylate polymerizations, which are extremely beneficial for dental resin applications. These advantages include: high gel-point conversion which significantly decreases shrinkage stress; rapid polymerization rate and lack of oxygen inhibition; nearly complete consumption of low molecular weight reacting species due to the nature of the step-growth mechanism, which limits the amount of leachable species and exhibiting less perceptible odor; versatile kinetics and structure-property design based on tailoring the thiol-ene monomer chemistry.

### EXAMPLES

Experimental work on the oligomeric thiol-ene systems as restorative materials was performed to demonstrate the feasibility and advantages of these polymers over currently used dental restorative materials. More specifically, the following polythiol monomers and polyvinyl monomers were utilized. 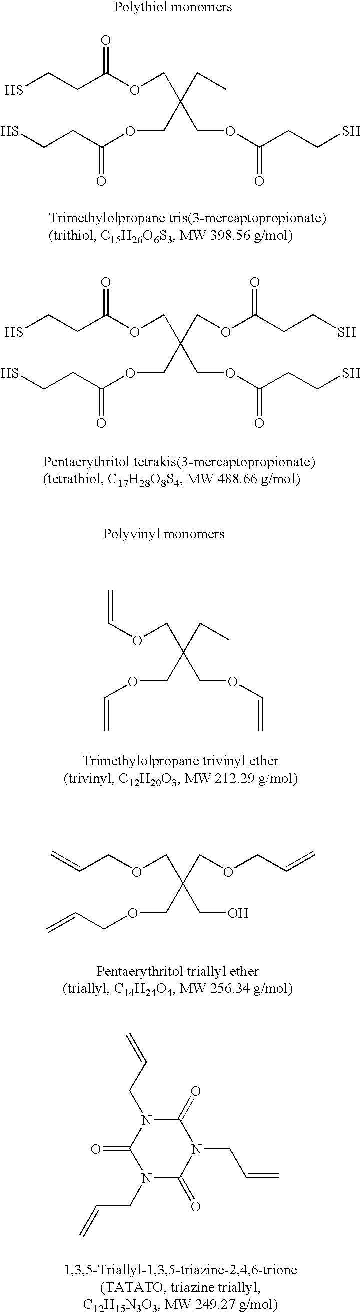
In addition, the following methacrylate system was used as a comparison:

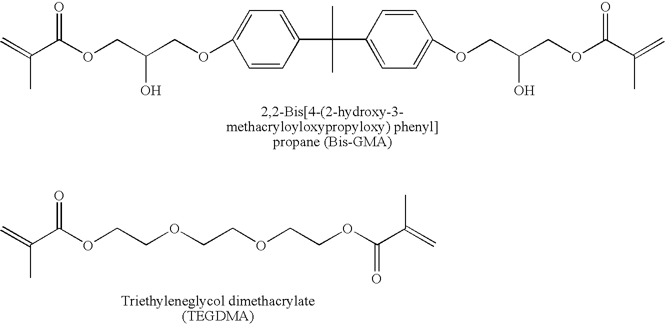

The thiol and vinyl monomers used in this investigation were triallyl-1,3,5-triazine-2,4,6-(1H,3H,5H)-trione (Triazine triallyl), Pentaerythritol triallyl ether (Triallyl), Trimethylolpropane trivinyl ether (Trivinyl), pentaerythritol tetra(3-mercaptopropionate) (tetrathiol) and trimethylolpropane tris(3-mercaptopropionate) (trithiol) (all obtained from Aldrich, Milwaukee, Wis.). The dimethacrylate monomers evaluated were 2,2-bis[p-(3-methacryloxy-2-hydroxypropoxy)phenyl]propane (Bis-GMA) and triethylene glycol dimethacrylate (TEGDMA) (Esstech, Essington, Pa.). Other materials include visible light photoinitiators camphorquinone (CQ) and ethyl 4-dimethylaminobenzoate (EDAB) (Aldrich) and 2,2-dimethoxy-2-phenylacetophenone (DMPA) (Ciba-Geigy, Hawthorn, N.Y.) was used as the UV photoinitiator. All monomers and photoinitiators were used without additional purification. The thiol-ene resins used in this study were prepared as stoichiometric mixtures based on equivalent functional group concentrations, whereas the Bis-GMA/TEGDMA resins were prepared as a 70/30 mass ratio, which is similar to the ratio used in commercial resins. Three samples per experimental composition were prepared for each test, using bulk resin (no filler) and 0.1 wt % DMPA as the initiator for UV light curing experiments, or 0.3 wt % CQ and 0.8 wt % EDAB as co-initiators for visible light experiments.

### Example 1

### Preparation and Conversion Analysis of Polythiol and Polyvinyl Oligomers

The purposes of synthesizing oligomeric thiol and ene materials are to optimize both polymer properties and polymerization performance and eliminate odor concerns. Because of the step growth nature of the thiol-ene photopolymerization, it is possible to oligomerize (both synthetic and commercially available) monomers to a significantly higher extent of polymerization prior to formulating the materials and completing the polymerization in the restoration. This technique is expected to have enormous advantages over the low molecular weight embodiments of the present invention studied herein. First, since the overall functional group concentration will be decreased dramatically, the shrinkage will correspondingly be decreased while still maintaining the identical ultimate network structure and material properties. Secondly, with higher molecular weight thiols, it will be more facile to purify the oligomers and remove the trace, low molecular weight compounds responsible for the odor in these systems and to limit further the amount of extractables.

By performing the photopolymerization (outside the cavity or body well before the material is needed) with an excess of either the vinyl or thiol functionality, it is possible to form highly functional, reactive non-gelled oligomers that are nearly exclusively one functional group terminated.

Polythiol monomer and polyvinyl monomers and DMPA for each oligomerization were added to a 20 mL scintillation vial and stirred magnetically on a coming stirplate using a 0.5 inch by 0.25 inch stirbar throughout the entire polymerization. The specific masses used for each oligomerization are given in Table 1.

TABLE 1
Mass amounts of polythiol and polyvinyl monomers and
DMPA used for each vinyl or thiol oligomer prepared

| Thiol | Vinyl | Mass | Mass | Mass | |
|---|---|---|---|---|---|
| Monomer | Monomer | Thiol, g | vinyl, g | Initiator, g | Oligomer Type |
| Trithiol | Triallyl | 0.47837 | 1.38395 | 0.00188 | vinyl oliqomer |
| Trithiol | Trivinyl | 0.25301 | 0.63064 | 0.00085 | vinyl oliqomer |
| Trithiol | triazine | 6.19939 | 0.86898 | 0.00751 | thiol oligomer |
| | Triallyl | | | | |
| Tetrathiol | Triazine | 2.3341 | 0.237 | 0.00252 | thiol oliqomer |
| | Triallyl | | | | |

Photoinduced oligomerization was conducted using a 365 nm light source (EFOS Ultracure 100 ss Plus) with an irradiation intensity at the surface of the sample of 80 mW/cm².

Conversion of the thiol and vinyl functional groups was monitored using FTIR (Magna 750, Nicolet Instrument Corp., Madison Wis.) because of its inherent advantage of being able to measure the thiol and vinyl conversions simultaneously and rapidly. The infrared peak at 1643 cm⁻¹ was used to determine the vinyl conversion, and the peak at 2572 cm⁻¹ was used for the thiol group conversion.

As a specific example, thiol oligomerization using the monomer functionality ratios mentioned above, results in r values (Equation 1) of 0.15 and 0.23 for the tetrathiol and trithiol oligomers, respectively, and consequently proportionally lowers the vinyl functional group concentration in the polymeric resins.

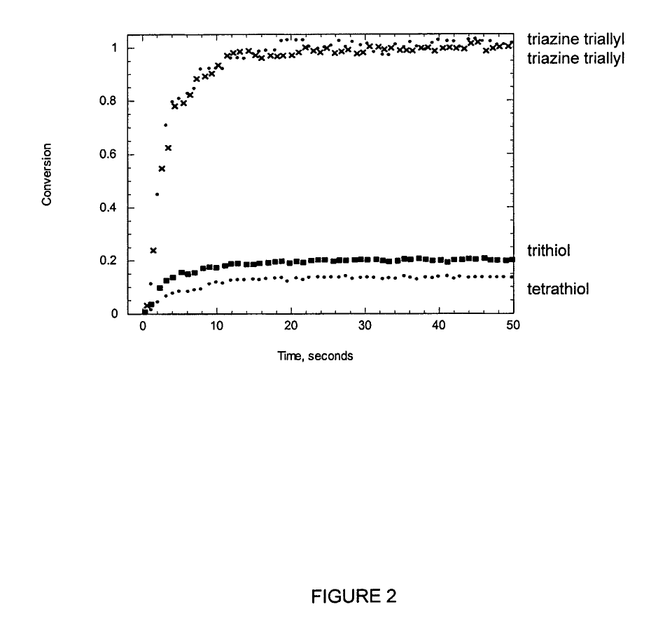

Trithiol/triazine triallyl and tetrathiol/triazine triallyl thiol terminated oligomer conversion for vinyl and thiol functional groups have been superimposed in FIG. 2. These preparations via the photopolymerization method created reactive thiol oligomers, such that the vinyl monomer is almost completely consumed, and the tetrathiol and trithiol react to the expected degree of conversion, as determined by Equation 1. The resulting multifunctional thiol-ene oligomers were used for both kinetic and mechanical evaluation. The prepared thiol-ene oligomers were stored unpurified and away from light sources at ambient conditions.

### Example 2

### Preparation and Testing of Thiol-ene System Formulations

Final formulations prepared using oligomers and monomers were made as stoichiometric mixtures based on equivalent functional group concentrations. All thiol-ene monomer-monomer, monomer-oligomer and oligomer-oligomer mixtures were prepared to have an equivalent concentration of thiol and vinyl functional groups. Oligomer functional group stoichiometry was determined by original monomeric amounts used in oligomer preparation adjusted for conversion as determined by FTIR.

For example, tetrathiol oligomer (0.35304 g) was combined with triazine triallyl (0.18427 g, 2.2 mmol CH═CH₂) and DMPA (0.00054 g) was used as the initiator. Three samples per experimental composition were prepared for each test using bulk resin with no filler and 0.1 wt % DMPA as the initiator for UV light curing experiments, or 0.3 wt % CQ as initiator with 0.8 wt % EDAB for visible light experiments.

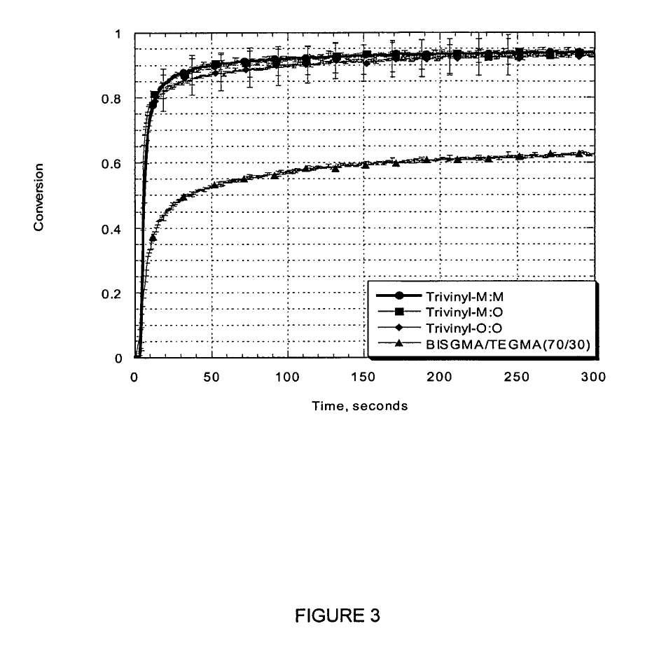

Conversion kinetics were measured via FTIR. Conversion of the vinyl functional group for Trithiol/Trivinyl (monomer:monomer, M:M), Trithiol/Trivinyl Oligomer (monomer:oligomer, M:O), Trithiol oligomer/Trivinyl oligomer (O:O), and Bis-GMA/TEGDMA (70/30 by wt.) are shown in FIG. 3 as a function of irradiation time; 0.1 wt % DMPA; UV=15 mW/cm² were used in this experiment. Conversion was greater than 90% for each thiol-ene polymerization, while the conventional Bis-GMA/TEGDMA (70/30 by wt.) exhibited approximately 63% vinyl conversion at 300 seconds. The thiol-ene monomer mixture in this experiment was prepared to have an equivalent concentration of the two functional groups.

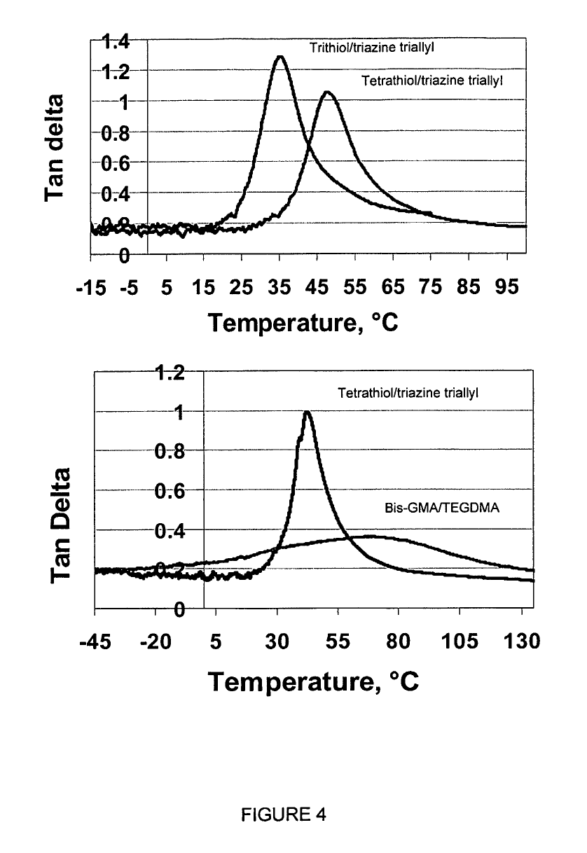

In addition to conversion kinetics, multiple material property measurements were conducted. Samples for dynamic mechanical analysis (DMA) using a DMA7e, Perkin-Elmer, Norwalk, Conn., were cured for 800 seconds using 15 mW/cm² UV light. DMA studies were conducted over a temperature range of −50 to 120° C., with a ramping rate of 5 ° C./min using extension mode (sinusoidal stress of 1 Hz frequency) and the loss tangent peak was monitored as a function of temperature. Tan δ (the ratio of loss to storage modulus) was monitored as a function of temperature. The loss tangent is defined as the polymer's loss modulus divided by storage modulus. During a DMA test, loss tangent peak corresponds to the viscoelastic relaxation of polymer chain or segments. Normally, the largest loss tangent peak can be associated with the polymer's glass transition peak and the temperature of the loss tangent peak maximum was used to define T_{g} (glass transition temperature). FIG. 4 illustrates T_{g} loss tangent peaks for thiol-ene systems trithiol/triazine triallyl and tetrathiol/triazine triallyl compared to Bis-GMA/TEGDMA. The Bis-GMA/TEGDMA exhibited a much broader peak width while the thiol-ene systems exhibited a narrower peak width indicative of a more homogenous network. The glass transition temperature (T_{g}) was taken to be the maximum of the loss tangent-temperature curve. Further T_{g} results for various thiol-ene systems are shown in Table 2.

Samples for flexural strength and elastic modulus investigation were prepared using steel molds measuring 2 mm×2 mm×25 mm and photocuring for 800 seconds using 15 mW/cm² UV light. Polymer flexural strength and modulus were calculated using a 3-point flexural test, carried out with a hydraulic universal test system (858 Mini Bionix, MTS Systems Corporation, Eden Prairie, Minn., USA) using a span width of 10 mm and a crosshead speed of 1 mm/min. The flexural strength (σ) and flexural modulus (E_{f}) in MegaPascals (MPa) were calculated using the following equations:

$$\begin{matrix}σ = \frac{3 Fl}{2 b {h}^{2}} & ( Equation 2 ) \\ {E}_{f} = \frac{{F}_{1} {l}^{3}}{4 b {h}^{3} d} & ( Equation 3 )\end{matrix}$$

where F is the peak load (in N), 1 is the span length (in mm), b is the specimen width (in mm), h is the specimen thickness (in mm); and d is the deflection (in mm) at load F₁ (in N) during the straight line portion of the trace (ISO/DIS 4049, 1987). ISO/DIS 4049 is the international standard for “Dentistry—Polymer-based filling, restorative and luting materials”. Flexural strength test is one of the tests specified in this standard for the polymer-based filling, restorative and luting materials.

The results in Table 2 show that while the mechanical properties of the current formulation are not as high as the current Bis-GMA/TEGDMA resin system, the flexural strength and the flexural modulus of the monomeric and oligomeric resins are not significantly different, and the Tgs of the oligomeric thiol-ene resins show a slight decrease compared to their monomeric thiol-ene counterparts.

Table 2. Glass transition temperature, flexural strength and flexural modulus measurements for Bis-GMA/TEGDMA (70/30 wt %) and nonfilled monomeric and oligomeric thiol-enes. Experiments were conducted at ambient temperature using 15 mW/cm² UV light, and 0.1 wt % initiator. Standard deviation in parentheses, n=3.

| | | Flexural | Flexural |
|---|---|---|---|
| | | Strength, | Modulus, |
| resin | Tg, ° C. | (MPa) | (GPa) |
| Trithiol: Triazine Triallyl | 33.8(1.3) | 22(3) | 0.15(0.02) |
| OligTrithiol: Triazine Triallyl | 29.9(1.3) | 17(1) | 0.13(0.01) |
| Tetrathiol: Triazine Triallyl | 49.0(1.6) | 76(8) | 1.70(0.20) |
| Olig Tetrathiol: Triazine Triallyl | 42.8(0.4) | 74(2) | 1.70(0.04) |
| Bis-GMA/TEGDMA(70/30 wt%) | 77.1(1.1) | 112(9) | 2.2(0.10) |

Simultaneous Measurement of Thiol-ene Shrinkage Stress and Conversion

This experimental set-up is capable of simultaneous measurement of the shrinkage stress and conversion, both on the same sample at the same time. The in situ, real-time monitoring of the polymerization was achieved by guiding the near-IR beam through the sample, which was mounted on the tensometer, then refocusing the transmitted signal to the near-IR detector. The tensometer, designed by American Dental Association (ADA), is based on the cantilever beam deflection theory: shrinkage force generated by the composite during curing causes the beam to bend, and the deflection is measured with a linear variable differential transformer (LVDT). The shrinkage force is then calculated using the beam constant of the cantilever beam. Therefore, the shrinkage stress value is obtained by dividing the shrinkage force by the composite sample cross-sectional area. With the combination of different beam lengths and materials, it is possible to measure the shrinkage stress accurately over a wide range of values. Using a tensometer designed by the American Dental Association, shrinkage stress was measured as a function of conversion. Stress development was monitored during cure as well as 10 minutes post cure. Samples measuring 6 mm in diameter and 2.5 mm in thickness and prepared using 0.3 wt % CQ and 0.8 wt % EDAB as initiator, were irradiated using a 400 mW/cm² (measured at the tip of the light guide) visible light source (Dentsply QHL CuringLite) for 60 seconds.

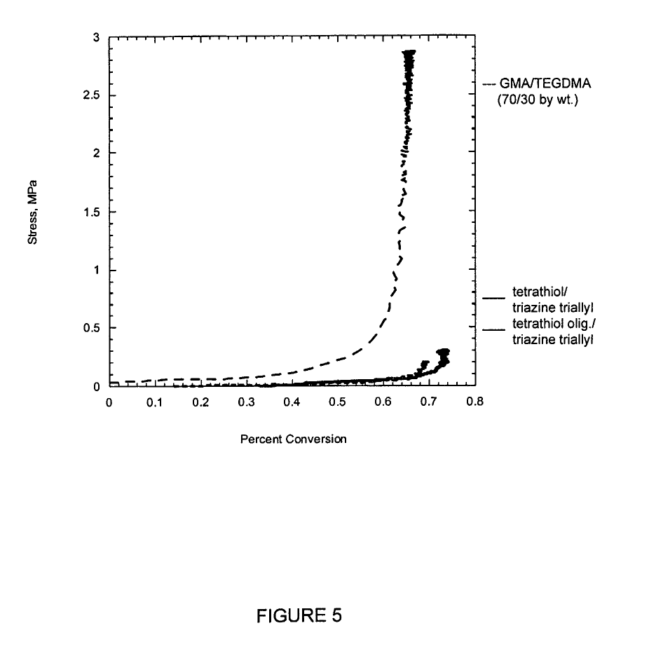

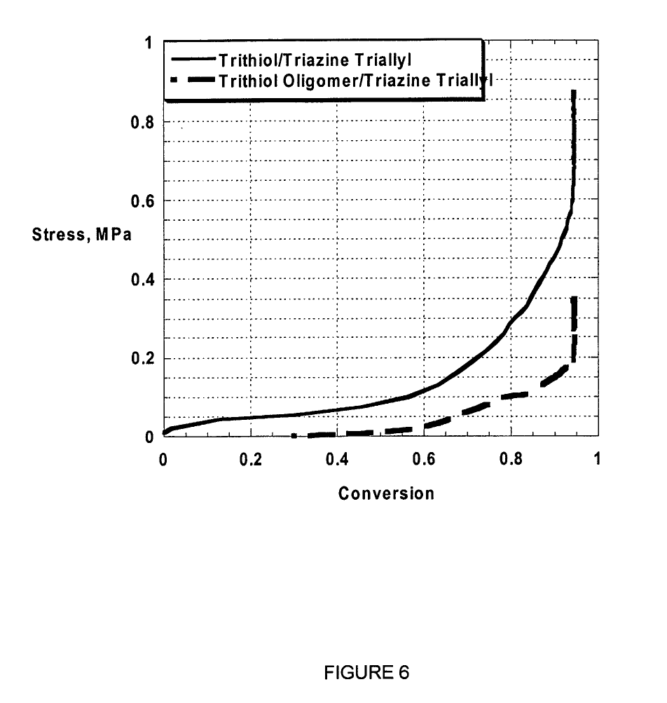

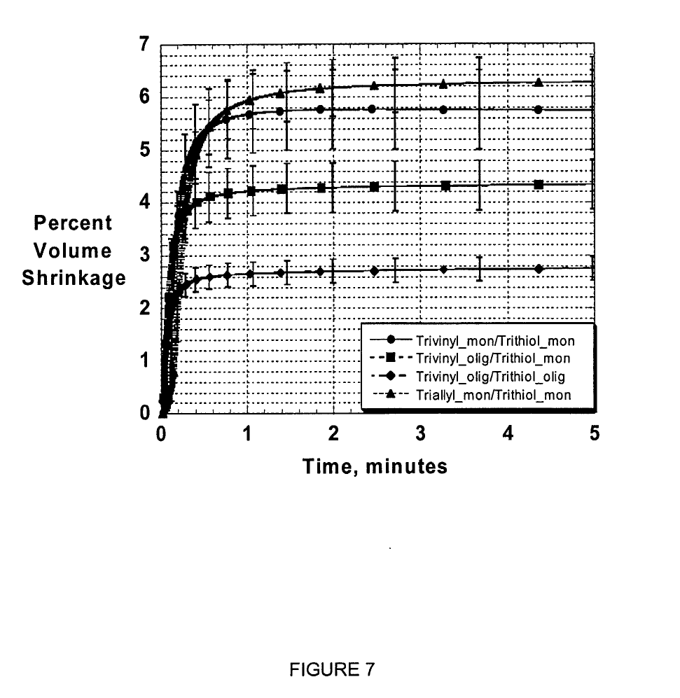

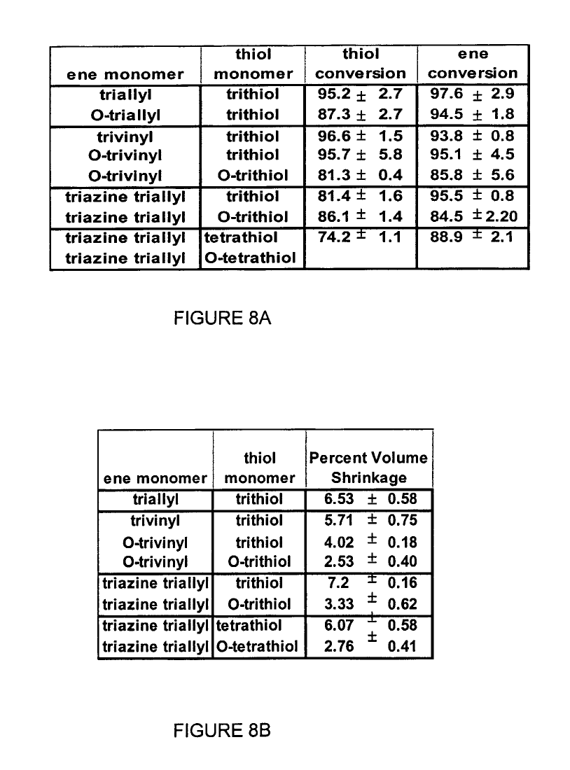

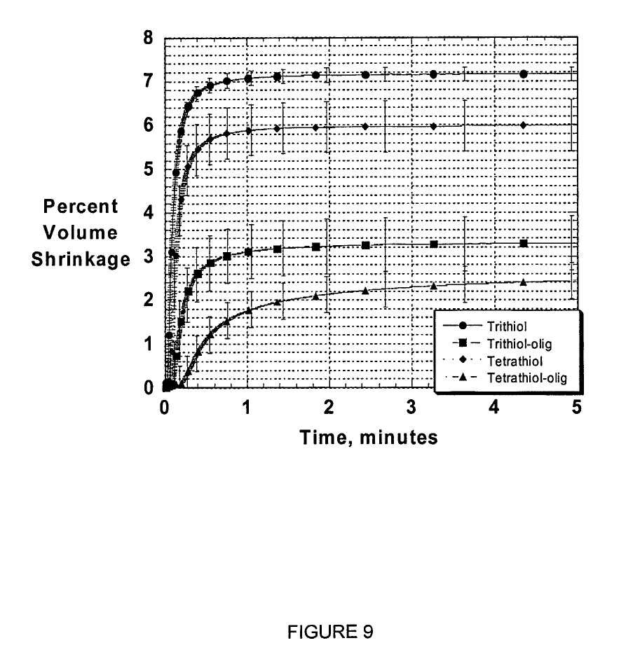

As seen in FIG. 5, the final shrinkage stress achieved by the tetrathiol

What is claimed is:

1. A dental composition comprising
a curable blend of one or more polythiol compounds and one or more polyvinyl compounds, wherein
said polythiol compounds are polythiol oligomers.

2. The dental composition of claim 1 wherein
said polythiol oligomers are formed by prepolymerization of first polyvinyl monomers in the presence of an excess of first polythiol monomers.

3. The dental composition of claim 1, wherein
said polyvinyl compounds are polyvinyl oligomers.

4. The dental composition of claim 3, wherein
said polyvinyl oligomers are formed by prepolymerization of second polythiol monomers in the presence of an excess of second polyvinyl monomers.

5. The dental composition of claim 1 further comprising at least one filler.

6. The dental composition of claim 5 further comprising at least one photoinitiator.

7. The dental composition of claim 6 wherein said at least one photoinitiator is selected from the group consisting of camphorquinone, ethyl 4-dimethylaminobenzoate, and 2,2-dimethoxy-2-phenylacetophenone.

8. The composition of claim 1 wherein said first polythiol monomers are chosen from the group consisting of trimethylolpropane tris(3-mercaptopropionate), and pentaerythritol tetrakis(3-mercaptopropionate).

9. The composition of claim 1 wherein said first polyvinyl monomers are selected from the group consisting of trimethylolpropane trivinyl ether, pentaerythritol triallyl ether, and 1,3,5-triallyl-1,3,5-triazine-2,4,6-trione.

10. The dental composition of claim 4 wherein the first polyvinyl monomer and the second polyvinyl monomer are different monomers.

11. The dental composition of claim 4 wherein the first polythiol monomer and the second polythiol monomer are different monomers.

12. The dental composition of claim 2 wherein the first polyvinyl monomer and the polyvinyl compound are different.

13. The dental composition of claim 2 wherein the first polythiol monomer is formed by the method comprising:
reacting a diisocyanate with an excess of an alcohol monomer to obtain a polyalcohol monomer;
converting one or more hydroxy groups on said polyalcohol monomer to thiol functional groups to obtain the polythiol monomer.

14. The dental composition of claim 2 wherein the polythiol monomer is formed by the method comprising:
reacting a diisocyanate with an excess of an alcohol monomer, wherein said alcohol monomer has at least one thiol functional group, to form the polythiol monomer.

15. The dental composition of claim 2 wherein the polyvinyl monomer is formed by the method comprising:
reacting a diisocyanate; in an excess of an alcohol monomer to form polyalcohol monomers having hydroxy functional groups;
reacting the polyalcohol monomers with vinyl acetate to form the polyvinyl monomers.

16. A dental composition comprising a curable blend of one or more polythiol compounds and one or more polyvinyl compounds, wherein
at least one of said polyvinyl compounds are polyvinyl oligomers.

17. The dental composition of claim 16, wherein
said polyvinyl oligomers are formed by prepolymerization of polythiol monomers in the presence of an excess of polyvinyl monomers.

18. The dental composition of claim 16 further comprising at least one filler.

19. The dental composition of claim 18 further comprising at least one photo initiator.

20. The dental composition according to claim 19 wherein said at least one photoinitiator is selected from the group consisting of camphorquinone, ethyl 4-dimethylaminobenzoate, and 2,2-dimethoxy-2-phenylacetophenone.

21. The composition of claim 17 wherein said polythiol monomers are chosen from the group consisting of trimethylolpropane tris(3-mercaptopropionate), and pentaerythritol tetrakis(3-mercaptopropionate).

22. The composition of claim 17 wherein said polyvinyl monomers are chosen from the group consisting of trimethylolpropane trivinyl ether, pentaerythritol triallyl ether, and 1,3,5-triallyl-1,3,5-triazine-2,4,6-trione.

23. The composition of claim 17 wherein the polythiol compound and the polythiol monomer are different.

24. A method of preparing a dental composition comprising the steps:
a. polymerizing first polyvinyl monomers in presence of an excess of first polythiol monomers to obtain polythiol oligomers;
b. polymerizing second polythiol monomers in presence of an excess of second polyvinyl monomers having vinyl functional groups to obtain polyvinyl oligomers; and
c. stoichiometrically mixing the polythiol oligomers and the polyvinyl oligomers to obtain a first mixture.

25. The method of claim 24 further comprising:
d. polymerizing the first mixture.

26. The method of claim 24 further comprising:
d. mixing the first mixture with at least one filler having color and at least one photoinitiator to obtain a second mixture.

27. The method of claim 26 further comprising:
e. packaging the second mixture in a container based on a color of the filler.

28. The method of claim 27 further comprising:
f. dispensing at least a portion of the second mixture from the container;
g. shaping the dispensed portion of the second mixture into a dental prosthesis; and
h. photopolymerizing the second mixture.

29. The method of claim 17 further comprising:
reacting a diisocyanate in an excess of an alcohol monomer to form polyalcohol monomers having hydroxy functional groups;
reacting the polyalcohol monomers with vinyl ethers to form the polyvinyl monomers of step (a); and
reacting the diisocyanate with an excess of an alcohol monomer, wherein said alcohol monomer has at least one thiol functional group, to form the polythiol monomers of step (a).

30. A method of preparing a shaped dental prosthetic device comprising the steps:
a. dispensing a mixture of one or more polythiol compounds and one or more polyvinyl compounds, wherein said polythiol compounds are polythiol oligomers formed by prepolymerization of first polyvinyl monomers in the presence of an excess of first polythiol monomers;
b. shaping the mixture into a dental prosthesis; and
c. polymerizing the mixture.

31. The method of claim 30 further comprising:
reacting a diisocyanate in an excess of an alcohol monomer to form polyalcohol monomers having hydroxy functional groups;
reacting polyalcohol monomers with vinyl ethers to form the polyvinyl monomers; and
reacting the diisocyanate with an excess of an alcohol monomers, wherein said alcohol monomer has at least one thiol functional group, to form the polythiol monomers.

32. The method of claim 30 wherein the mixture further comprises a filler and the method further comprises: selecting the mixture based on filler color.

33. The method of claim 32 wherein the mixture includes at least one photoinitiator and polymerizing further comprises:
photopolymerizing the mixture by exposing it to a light source operable to cause the photoinitiator to initiate the polymerization reaction.

34. The method of claim 30, wherein
said polyvinyl compounds of step (a) are polyvinyl oligomers formed by prepolymerization of second polythiol monomers in the presence of an excess of second polyvinyl monomers.

35. The method according to claim 33 wherein said at least one photoinitiator is selected from the group consisting of camphorquinone, ethyl 4-dimethylaminobenzoate, and 2,2-dimethoxy-2-phenylacetophenone.

36. The method of claim 30 wherein said first polythiol monomers are chosen from the group consisting of trimethylolpropane tris(3-mercaptopropionate), and pentaerythritol tetrakis(3-mercaptopropionate).

37. The method of claim 30 wherein said first polyvinyl monomers are chosen from the group consisting of trimethylolpropane trivinyl ether, pentaerythritol triallyl ether, and 1,3,5-triallyl-1,3,5-triazine-2,4,6-trione.

38. The method of claim 34 wherein the first polyvinyl monomer and the second polyvinyl monomer are different monomers.

39. The method of claim 34 wherein the first polythiol monomer and the second polythiol monomer are different monomers.

40. The method of claim 30 wherein the first polyvinyl monomer and the polyvinyl compound are different.
